(19) United States
(12) Patent Application Publication
(10) Pub. No.: US 2008/0058789 A1
(43) Pub. Date: Mar. 6, 2008

(54) GUIDANCE SYSTEM USED IN TREATING CHRONIC OCCLUSION

(75) Inventor: Gary Roubin, Jackson, WY (US)

Correspondence Address: MAYER BROWN LLP, P.O. BOX 2828, CHICAGO, IL, 60690, US

(73) Assignee: CardioFirst, New York, NY (US)

(21) Appl. No.: 11/850,593

(22) Filed: Sep. 5, 2007

Related U.S. Application Data
(60) Provisional application No. 60/824,723, filed on Sep. 6, 2006.

(51) Int. Cl. A61B 18/18; A61B 1/00

(52) U.S. Cl. 606/15; 601/2; 604/20; 606/33

(57) ABSTRACT

Structures and methods for guidance of a guide wire in a blood vessel are disclosed. The guide wire comprises a metal housing having a lumen and four or more optical fibers contained within the lumen. The methods comprise obtaining multiple signals via the guide wire and processing said signal to obtain real-time positional information as to the location of the tip in the cross-section of the blood vessel.

### FIELD OF THE INVENTION

This invention relates generally to the field of guidance of a guide wire used in ducts in the human body which are subject to occlusion, and more specifically to the steering of such guide wires in addressing an obstruction in a blood vessel or other duct.

### BACKGROUND OF THE INVENTION

A theromatous plaque forming within the lumen of elastic arteries in the human body is well known. When such a plaque occupies more than 70 percent of the lumen it causes a reduction in the flow of oxygenated blood through the affected artery and eventually could produce debilitating or devastating outcomes such as heart attack, angina, or shortness of breath on exertion. When such plaque is formed within the lumen of the arteries that supply oxygenated blood to the heart tissue, it is called coronary artery disease.

Treatment options for such occlusions could include coronary angioplasty with deployment of a stent to strengthen the affected arterial wall, or even coronary artery bypass operation if coronary angioplasty fails, especially if the occlusion is total (i.e. covering 100% of the luminal surface), if it is organized, calcified, and is chronic in nature. When such occlusions occur for over 90 days and the lumen is totally occluded, they are often called chronic total occlusions. Using angioplasty to treat such occlusions is very challenging because the course of the artery, its size, and the length of such an occlusion are not known. As such, while it is possible for an operator to try to cross such an occlusion with a guide wire, the uncertainty of the boundaries may lead the operator to accidentally perforate the arterial wall with the guide wire. Complications from a perforation include hemorrhage, percardial tamponade and hemodynamic collapse, or compartment syndrome. Chronic occlusions can also occur in non-vascular structures, such as the common bile duct.

Substantial technology has been developed to diagnose the nature of such occlusions. These have included technologies which are able to provide images of the interior of a vessel in the vicinity of an occlusion. Among these have been techniques involving Optical Low Coherence Reflectometry (OLCR). For instance U.S. Pat. No. 5,582,171 teaches using an internal rotating mirror to sweep the interior of a vessel with a beam of light while U.S. Pat. No. 6,463,313 teaches the insertion of multiple optical fibers each with its own prism to obtain imaging signals from different portions of a cross section of a vessel interior. While these technologies provide signals from different portions of the cross section of a vessel there is no teaching or suggestion to process these signals to obtain the location in the vessel cross section of the guide wire tip (or “effector”) from which they emanate, let alone doing so by creating a vessel image and “effector” image that allows the operator to guide the “effector” device towards the center of the vessel.

Other technology has been developed to address the problem of avoiding puncture of a blood vessel wall when using a guide wire to deliver ablating energy to an obstruction. U.S. Pat. No. 6,852,109 teaches using OLCR to sense when a guide wire is in close proximity to a vessel wall. However, this system utilizes a single signal which simply provides an indication of the distance to the vessel wall but no other information on the relative position of the guide wire tip from which the signal emanates in the cross section of the vessel. The device is programmed to only warn the operator when the OCR signals indicate that the tip of the wire is too close to the circumferential limits of the tubular structure. However, the warning is either auditory in nature (as in a ‘beep’) or typically provides a single distance from the edge and thus provides only the information that the tip is too close to the vessel wall without any directional information. This therefore requires more time and care on the part of the operator in moving the wire, as the operator must guess as to which direction would most quickly return the wire to the center of the vessel. It therefore would be greatly helpful if there were a device or method that would yield a visual representation showing the actual relative position of the tip in the cross section of the vessel, particularly if it were shown in real time or near-real time.

### SUMMARY OF THE INVENTION

The present invention is concerned with apparatus and techniques for navigating through an occlusion in a duct, preferably a blood vessel, of the human body with a guide wire in which the position of the tip of the wire in the cross section of the vessel is ascertainable in real time as the wire is navigated through the occlusion. In a preferred embodiment the position of the tip is determined through the use of optical fibers carried by the guide wire. In a further preferred embodiment the guide wire carries at least three optical fibers. In a particularly preferred embodiment the guide wire carries at least four optical fibers. In a further preferred embodiment three optical fibers are arranged in a triangular configuration. In a particularly preferred embodiment of this triangular configuration a fourth optical fiber bundle is provided in the center of this triangular configuration. In a further preferred embodiment this optical fiber bundle is either a single optical fiber or a group of four optical fibers which are arranged in a square configuration.

In a further preferred embodiment of determining the tip position using optical fibers infrared radiation is passed through the optical fibers. In a further preferred embodiment of determining the tip position using optical fibers radiation is passed through the fibers to reflect of the wall of the vessel and the reflected signals are processed by a dual processor CPU. In a further preferred embodiment the reflected signals from the optical fibers is processed in accordance with Optical Low Coherence Reflectometry (OLCR). In another preferred embodiment the real time position of the guide wire tip is displayed on a screen, preferably within a representation of the cross section of the vessel. In another preferred embodiment the guide wire carries both optical fibers and an ultrasonic probe. In a further preferred embodiment the ultrasonic probe is used to deliver energy to aid in disrupting the occlusion and preferably also sensing the nature of the occlusion.

In another preferred embodiment the guide wire carries optical fibers and one or more of these fibers is used to deliver energy, preferably laser generated energy, to aid in disrupting the occlusion. In a related preferred embodiment the optical fibers are used to generate signals which are used to steer the guide wire which is then used to deliver radio frequency energy to disrupt the occlusion.

In a particularly preferred embodiment the guide wire carries multiple optical fibers, each of which is adapted to deliver an optical signal to the vessel wall at a different radial angle than the other optical fibers and these signals are used to establish the position of the tip of the guide wire in the cross section of the vessel.

### BRIEF DESCRIPTION OF THE DRAWINGS

FIG. 1 is a view of an occluded vessel where a guide wire has successfully crossed the occlusion and the tip is within the distal lumen of the occluded vessel.

FIG. 2a is a longitudinal view of the guide wire showing the optical fibers within the guide wire

FIG. 2b is a cross-sectional view of the guide wire

FIG. 2c is an embodiment of the internal optical fiber where the internal fiber is a grouping of four optical fibers

FIG. 3 is an illustration of a desirable tip or “effector” position

FIG. 4 is a view of one embodiment of the display of the interior of a vessel, showing OLCR reference points and the location of the “effector” or tip within the vessel.

### DETAILED DESCRIPTION OF THE INVENTION

While the present invention is capable of embodiment in various forms, hereinafter is described an embodiment with the understanding that the present disclosure is to be considered as an exemplification of the invention, and is not intended to limit the invention to the specific embodiment illustrated.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of cardiology and pharmaceutical sciences or the art relevant to the range or element at issue. The amount of broadening from the strict numerical boundary depends upon many factors. For example, some of the factors to be considered may include the criticality of the element and/or the effect a given level of variation will have on the performance of the claimed subject matter, as well as other considerations known to those of skill in the art. Thus, as a general matter, “about” or “approximately” broaden the numerical value, yet cannot be given a precise limit. For example, in some cases, “about” or “approximately” may mean ±5%, or ±10%, or ±20%, or ±30% depending on the relevant technology. Also, the disclosure of ranges is intended as a continuous range including every value between the minimum and maximum values.

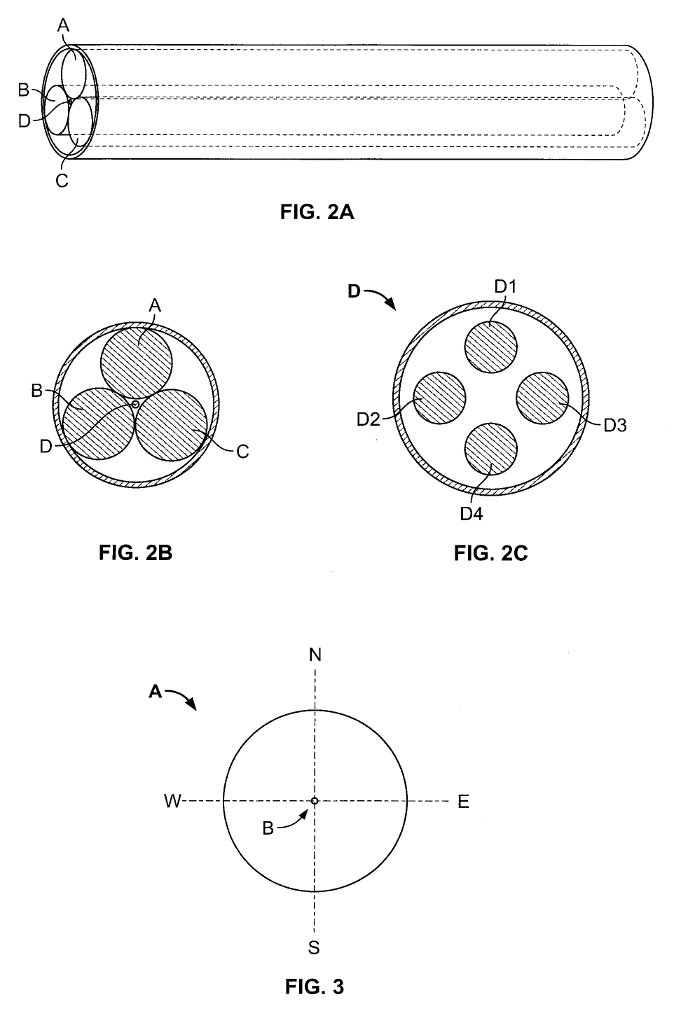

In one embodiment the present invention comprises a guide wire. FIG. 2a shows a longitudinal view of one embodiment of the guide wire. The guide wire has a metal housing. The housing has an internal lumen diameter of approximately 0.006 inch and a thickness of approximately 0.004 inch. The housing may be of any desirable length, but the presently preferred length is between 280 and 320 cm in length. It may be constructed of any suitable metal or alloy. In one embodiment the metal may be commercially pure titanium, stainless steel, or any other suitable metal as known to one skilled in the art.

Within the lumen of the housing, there are four or more optical fibers. Optical fibers A, B and C are a first group of optical fibers. In one embodiment, fibers A, B and C are the same diameter and are each approximately 80 microns in diameter, arranged in a triangular formation. In some embodiments the triangle may be an isosceles triangle. In a presently preferred embodiment, the triangle is approximately equilateral.

The three fibers emit light of near-infrared wavelengths. At the end of the three fibers is a device that deflects the beam outward radially toward the vessel wall. The device may be a mirror, such as one used in U.S. Pat. No. 5,582,171. It may also be a prism, such as one used in U.S. Pat. No. 6,463,313. Other suitable devices as known to one skilled in the art may also be used.

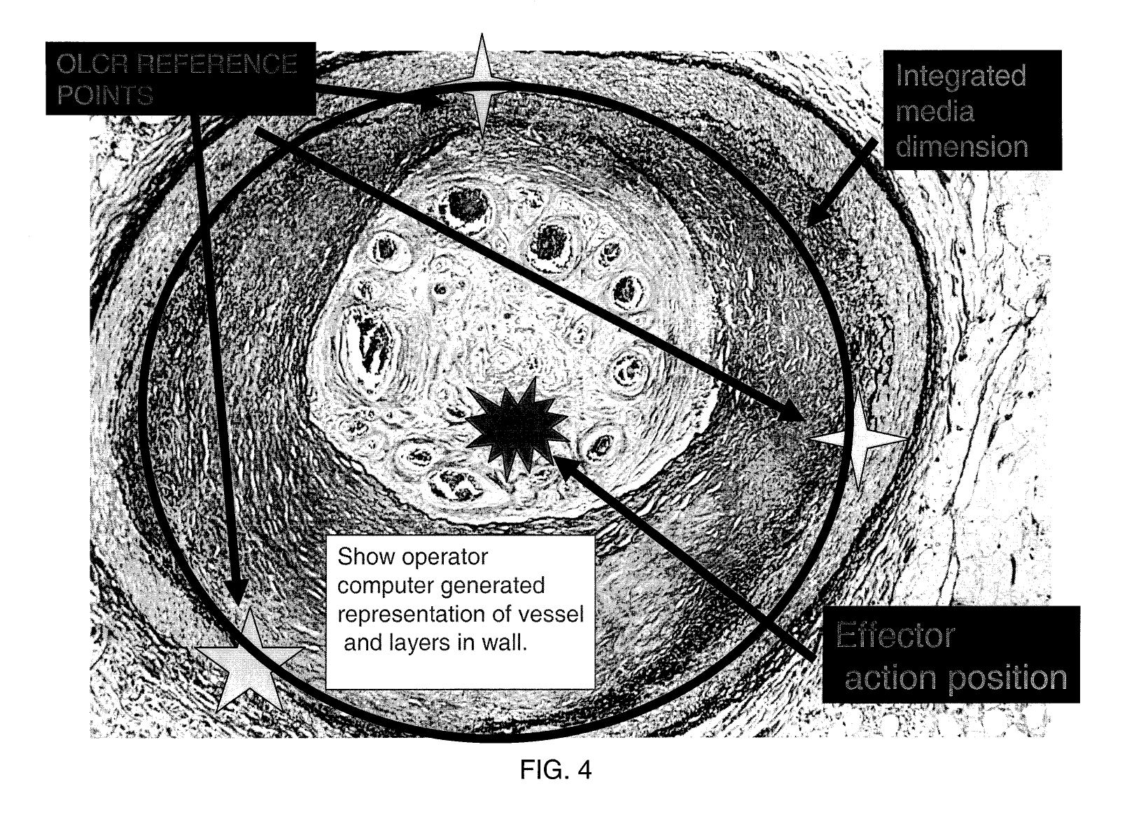

The light from each of the three fibers reflects off of the vessel wall and is communicated to the interferometer, thereby allowing for the distance of the tip from the three points on wall to be determined. A simple triangulation calculation therefore will allow for a visual depiction of the current position of the tip of the wire. As the wire is advanced, the operator will see the movement of the tip relative to the wall of the vessel in real time, and as such will be able to alter the direction of advancement of the wire to avoid contacting the vessel wall with the tip and therefore minimizing the possibility of rupture or perforation of the vessel. FIG. 3 shows a desirable position for the tip or “effector” of the guide wire. At position B, it is centered relative to the circumference of the arterial wall A. FIG. 4 is an example of one possible embodiment of the real-time image produced by one embodiment of the invention, further showing the three reference points on the wall where the light from the three fibers reflects and the position of the “effector” or tip within the vessel.

A second group of optical fibers D is also contemplated. The second group may consist of a single optical fiber that is approximately 10 to 20 microns in diameter. FIG. 2b shows a cross-section of the guide wire and shows optical fibers A, B, C and D. In another embodiment, the second group of optical fibers D may be four optical fibers D1, D2, D3 and D4, as shown in FIG. 2c. Such optical fibers are arranged within the triangular formation of fibers A, B and C and have diameters of approximately 5 microns each. The second group of fibers D regardless of number of fibers is separately insulated from fibers A, B and C. These fibers may be used to emit beams of light that are directed at the vessel wall and the reflections used in OLCR to generate signals which can be processed to locate the guide wire tip in the cross section of the vessel in a manner similar to that described for the outer group of fibers. These beams may be utilized to determine distance from an occlusion in real time. For example, as discussed above the data from the beams of the first group of fibers will allow the operator to know if the wire is approaching the vessel wall. The beam or beams emitted from the second group of fibers will detect the distance in front of the wire before a solid structure is contacted. If the wire is being moved in the center of the vessel and yet the distance detected by the second group of fibers continues to shrink and approach zero, then the operator will know that the occlusion is approaching. In the case of a single central fiber it may be used to deliver optical energy such as laser generated light to aid in disrupting the occlusion.

Where an occlusion is found, in one embodiment the first set of fibers may be used to emit energy at the occlusion in order to assist in dissipation and/or ablation of the occlusion. In another embodiment, the second group of fibers may be used to emit such energy. In a further embodiment, both sets of fibers may be used. In still another embodiment, only certain fibers from the first, second or both groups are used. In any such embodiment, the energy used may be of any type readily transmitted by an optical fiber such as laser generated visible or infrared light. In addition the disruptive energy may be partially or wholly supplied by the guide wire itself, such as radio frequency energy, or by an auxiliary device such as an ultrasonic probe mounted on the guide wire.

It is further contemplated that a small balloon may be incorporated near the tip of the guide wire to help in its centering in the vessel before the guide wire enters the occlusion. The balloon would be inflated, and upon its expansion to the walls of the vessel, the wire would thereby become approximately centered in the vessel.

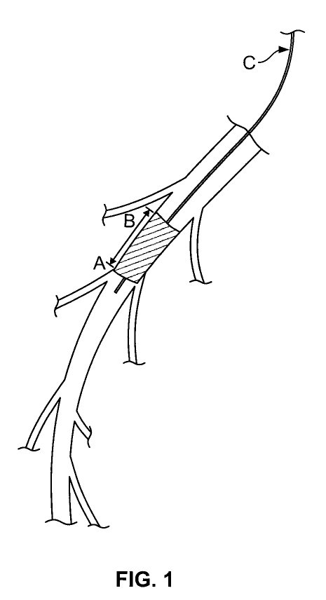

Once the guide wire enters into the occlusion it should be understood that the “vessel wall” which is sensed may, in fact, be the wall of the passage through the occlusion which the guide wire creates by providing disruptive energy to the occlusion. This would be the case when the occlusion is not entirely disrupted but remains as a plaque on the wall of the vessel. FIG. 1 shows a view of an occluded vessel where a guide wire has successfully crossed the occlusion and the tip is within the distal lumen of the occluded vessel.

In order to aid its navigation through organized, highly resistant or calcified plaque, in some embodiments the distal end of the wire may have an ultrasound probe (not shown). The ultrasound probe is of approximately 0.004 inch in thickness and 4 mm in length, and is attached to the distal portion of the wire approximately 0.2 mm from its protruded tip. The ultrasound probe operates at a high frequency, creating vibrational energy that will aid in dissipation of the calcified area and thereby creating a path of lesser resistance for the wire to be advanced. The ultrasound probe also may also be used to provide information regarding the composition of the plaque and thereby aid in the selection of devices for treatment of such a diseased segment. Furthermore, the ultrasound probe may allow the operator to assess the efficacy of his efforts and to ensure that any stent or stents used are properly and optimally positioned before the guide wire is removed.

In some embodiments, the guide wire further comprises an additional “sleeve” to provide additional strength and stiffness to the tip in aid of the navigation process. In one embodiment, the sleeve is constructed of stainless steel. In one embodiment, the sleeve has a thickness of 0.0025 inch and an inner diameter of 0.0145 inch, allowing the sleeve to fit over the distal end of the guide wire. The inner diameter of the sleeve should be chosen so as to be slightly larger than the diameter of the guide wire mechanism. The sleeve may be moved slightly forward or backward to allow the position of the tip to be determined as discussed above.

The supporting apparatus includes an OLCR interferometer, a computer or other processing device to process signals, a device to control operation such as switches, knobs, or the like, and a display monitor to display the images generated by the computer from the data obtained via the OLCR method from the light emitted through the wire. Such devices are known in the art. The computer presently preferred has a dual processor with substantial memory and may be programmable to deliver a selected amount of a selected type of energy as ordered by the operator. However, other computers are also usable in the present invention. The display monitor is preferably a high resolution monitor, although other monitors may be used.

Other techniques of imaging may be utilized so long as they are capable of providing multiple independent signals reflective of the distance from the guide wire from which they emanate to the “vessel wall”. For instance the MRI imaging techniques described in U.S. Pat. Nos. 6,549,800 and 6,675,033 may be adaptable to providing signals which can provide real time information on the position of the tip of a guide wire with which the MRI probe is associated. Another alternative is the ultrasonic imaging technology described in U.S. Pat. No. 5,916,210. In general any technology for imaging the interior of a vessel wall should be adaptable to providing guidance by appropriate processing of the signals which the technology already provides.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles. All patent and literature references are hereby incorporated by reference as if fully set forth herein.

What is claimed is:

1. A guide wire, comprising:
A metal housing having a lumen; and
four or more optical fibers in the lumen.

2. The guide wire of claim 1, wherein the metal of the housing is selected from the group consisting of commercially pure titanium or stainless steel.

3. The guide wire of claim 2, wherein the metal is commercially pure titanium.

4. The guide wire of claim 1, wherein the metal housing has a diameter of about 0.006 inch.

5. The guide wire of claim 1, wherein there are four optical fibers in the lumen

6. The guide wire of claim 5, wherein one optical fiber is in the center of the lumen.

7. The guide wire of claim 6, wherein three optical fibers are arranged in a triangular formation about the center of the lumen.

8. The guide wire of claim 7, wherein the triangular formation approximates an isosceles triangle or an equilateral triangle.

9. The guide wire of claim 1, wherein there are seven optical fibers in the lumen.

10. The guide wire of claim 1, wherein the optical fibers have approximately the same diameter.

11. The guide wire of claim 1 further comprising a additional sleeve of metal added to the leading end of the guide wire.

12. The guide wire of claim 11, wherein the sleeve has a rotate and lock mechanism

13. The guide wire of claim 11, wherein the sleeve has a thickness of about 0.002 inch.

14. A directional guidance system for a guide wire, comprising:
the guide wire comprising:
a) metal housing having a lumen; and
b) four or more optical fibers in the lumen;
an interferometer in communication with the guide wire;
a computer in communication with the interferometer; and
a display monitor connected to the computer.

15. The system of claim 14, wherein the metal of the housing of the guide wire is selected from the group consisting of commercially pure titanium or stainless steel.

16. The system of claim 15, wherein the metal is commercially pure titanium.

17. The system of claim 14, wherein the metal housing has a diameter of about 0.006 inch.

18. The system of claim 14, wherein there are four optical fibers in the lumen

19. The system of claim 18, wherein one optical fiber is in the center of the lumen.

20. The system of claim 19, wherein three optical fibers are arranged in a triangular formation about the center of the lumen.

21. The system of claim 20, wherein the triangular formation approximates an isosceles triangle or an equilateral triangle.

22. The system of claim 14, wherein there are seven optical fibers in the lumen.

23. The system of claim 14, wherein the optical fibers have approximately the same diameter.

24. The system of claim 14 further comprising a additional sleeve of metal added to the leading end of the guide wire.

25. The system of claim 24, wherein the sleeve has a rotate and lock mechanism

26. The system of claim 24, wherein the sleeve has a thickness of about 0.002 inch.

27. The system of claim 14 further comprising an ultrasound probe adjacent to the distal end of the guide wire.

28. The system of claim 14, wherein the interferometer is an OLCR interferometer.

29. The system of claim 14, further comprising an energy source that emits energy of the type selected from the group consisting of heat, laser, optical, radio frequency and combinations thereof.

30. The system of claim 14, wherein the computer is a dual process computer.

31. A directional guidance system for a guide wire, comprising:
the guide wire comprising:
a) metal housing having a lumen;
b) at least one optical fiber in approximately the center of the lumen; and
c) a second group of three optical fibers in a triangular shape around the center of the lumen;
an OLCR interferometer in communication with the guide wire;
a computer in communication with the interferometer; and
a display monitor connected to the computer.

32. A directional guidance system for a guide wire, comprising:
the guide wire comprising:
a) a housing constructed of commercially pure titanium, said housing having a lumen;
b) a first group of four optical fibers arranged in an approximate square shape in approximately the center of the lumen; and
c) a second group of three optical fibers in a triangular shape around the center of the lumen and surrounding the first group of optical fibers;
an OLCR interferometer in communication with the guide wire;
a computer in communication with the interferometer; and
a display monitor connected to the computer.

33. A method for efficiently passing a guide wire through an occluded segment of a blood vessel while minimizing the risk of perforating the wall of said vessel comprising obtaining multiple signals each emitted from the guide wire at a different radial angle and providing information as to the distance of the guide wire from the wall of said vessel, processing said multiple signals so as to obtain the real time position of the tip of said guide wire in the cross section of said vessel and using said positional information to advance said guide wire through said occlusion.

34. The method of claim 33 in which the guide wire contains means, including multiple independent optical fibers, configured to emit electromagnetic radiation directed at the wall of said blood vessel at multiple radial angles.

35. The method of claim 34 in which the guide wire includes at least three optical fibers.

36. The method of claim 33 in which the guide wire includes means to deliver energy to the occlusion so as to disrupt it.

37. The method of claim 36 in which the disruptive energy is electromagnetic radiation or ultrasound radiation.

38. The method of claim 37 in which the electromagnetic radiation is laser derived optical radiation.

39. The method of claim 37 in which the disruptive electromagnetic radiation is radio frequency radiation.

40. The method of claim 37 in which the guide wire contains means, including multiple independent optical fibers, configured to emit electromagnetic radiation directed at the wall of said blood vessel at multiple radial angles and one or more of said optical fibers is also used to deliver said disruptive electromagnetic radiation.

41. The method of claim 36 in which the guide wire includes an ultrasonic probe which generates high frequency vibrational energy which is used to disrupt said occlusion.

42. The method of claim 33 in which the cross sectional position of the tip of the guide wire is displayed on a screen which has a representation of the cross section of said blood vessel.

43. The method of claim 33 in which said multiple signals are processed by a dual processor CPU or multiple CPU's.

44. The process of claim 33 in which the cross sectional position of the tip of said guide wire is determined using Optical Low-Coherence Reflectometry.

45. The process of claim 33 in which said multiple signals include an infrared signal.

46. The method of claim 33 wherein the multiple signals are processed to yield a representational image of the cross section of said vessel showing the vessel wall and the tip of said guide wire.

47. A method of visualizing the position of a guide wire tip in a blood vessel comprising obtaining multiple signals which provide information as to the distance of said guide wire tip from different portions of the vessel wall by emitting radiation from said guide wire tip and receiving back reflections of said radiation from different portions of said vessel wall and processing said signals so as to create a representational image on a display device which shows the vessel wall and said guide wire tip.
